(19) United States
(12) Patent Application Publication
(10) Pub. No.: US 2011/0159484 A1
(43) Pub. Date: Jun. 30, 2011

(54) APPARATUS AND METHOD OF AUTHENTICATING PRODUCT USING POLYNUCLEOTIDES

(75) Inventors: Joo-won RHEE, Yongin-si (KR); Kyung-hee PARK, Seoul (KR)

(73) Assignee: SAMSUNG ELECTRONICS CO., LTD., Suwon-si (KR)

(21) Appl. No.: 12/859,868

(22) Filed: Aug. 20, 2010

(30) Foreign Application Priority Data
Dec. 30, 2009 (KR) 10-2009-0134927

(51) Int. Cl. C12Q 1/68; C12M 1/34

(52) U.S. Cl. 435/6; 435/287.2

(57) ABSTRACT

A method of authenticating a product includes; hybridizing a target polynucleotide which is associated with the product and which includes a first target region and a second target region adjacent to the first target region with a probe polynucleotide that comprises a first segment having a nucleotide sequence completely complementary to the first target region and a second segment having a nucleotide sequence completely complementary to the second target region, ligating one end of the first segment of the probe polynucleotide to one end of the second segment of the probe polynucleotide, amplifying the probe polynucleotide and detecting the amplified polynucleotide.

### CROSS-REFERENCE TO RELATED APPLICATION

This application claims priority to Korean Patent Application No. 10-2009-0134927, filed on Dec. 30, 2009, and all the benefits accruing therefrom under 35 U.S.C. §119, the contents of which in its entirety are herein incorporated by reference.

### BACKGROUND

1. Field

The present disclosure relates to an apparatus and method of authenticating a product using a polynucleotide.

2. Description of the Related Art

Counterfeiting products considerably damages legitimate producers by causing loss of sales, customers' distrust in the products and the perceived manufacturers of those products, compensation claims for counterfeit products, etc. It has been reported that loss caused by counterfeit and pirated products could be up to US $200 billion worldwide, and more than 10% of international trades is counterfeit and pirated. In addition, it is estimated that the losses of pharmaceutical companies caused by counterfeit drugs could be more than US $32 billion. Thus, there is a need to develop a method of authenticating a product to protect the legitimate producers and customers who trust the producers and purchase their products.

The use of a radio-frequency identification (“RFID”) tag is one method that has been used to authenticate a product. The RFID tag is embedded in a product and includes an identification code and brief information on the product. However, the security of the RFID tag is vulnerable. In addition, since the information corresponding to a product is easily obtained using only the identification code of the product, the information is not reliable. In particular, a counterfeit product having a RFID tag having a copied or counterfeited code of a genuine product may be incorrectly determined to be a genuine product.

In order to authenticate a product, a label such as a 3D hologram, a bar code, and a watermark has been attached to the product. However, there is no way for a customer to identify whether the label is genuine, and it is difficult for a user to easily authenticate a product without a specialized scanner. Furthermore, even though such a label has authentication information, the quantity of the information is limited. Thus, the authentication of a product may not be accurately conducted due to vulnerable security and difficulty in obtaining information on a distribution channel or history of the product.

### SUMMARY

Provided are an apparatus and method of authenticating a product using a polynucleotide.

An embodiment of a method of authenticating a product includes; hybridizing a target polynucleotide which is associated with the product and which includes a first target region and a second target region adjacent to the first target region with a probe polynucleotide that comprises a first segment having a nucleotide sequence completely complementary to the first target region and a second segment having a nucleotide sequence completely complementary to the second target region, ligating one end of the first segment of the probe polynucleotide to one end of the second segment of the probe polynucleotide, amplifying the probe polynucleotide and detecting the amplified polynucleotide.

An embodiment of an apparatus for authenticating a product includes; a sample inlet comprising a probe polynucleotide that comprises a first segment having a nucleotide sequence completely complementary to a first target region of a target polynucleotide and a second segment having a nucleotide sequence completely complementary to a second target region of a target polynucleotide and a ligase, wherein the first target region and the second target region of the target polynucleotide are adjacent to one another, an amplification unit comprising a polynucleotide polymerase, a primer, and dNTP and a detection unit which detects the amplified polynucleotides.

Additional aspects will be set forth in part in the description which follows and, in part, will be apparent from the description, or may be learned by practice of the presented embodiments.

According to an aspect of the present invention, an apparatus and method of authenticating a product using a polynucleotide are provided.

### BRIEF DESCRIPTION OF THE DRAWINGS

These and/or other aspects will become apparent and more readily appreciated from the following description of the embodiments, taken in conjunction with the accompanying drawings of which:

FIG. 1 schematically illustrates an embodiment of an apparatus for authenticating a product according to the present disclosure;

FIG. 2 schematically illustrates a hybridization according to an embodiment of a method of authenticating a product according to the present disclosure;

FIG. 3 illustrates an embodiment of a sequence of a target polynucleotide according to the present disclosure;

FIG. 4 illustrates an embodiment of a sequence of a probe polynucleotide according to the present invention; and

FIG. 5 is a graph illustrating the results of amplification according to an embodiment of the present disclosure.

### DETAILED DESCRIPTION

Embodiments now will be described more fully hereinafter with reference to the accompanying drawings, in which embodiments of the invention are shown. The embodiments may, however, be embodied in many different forms and should not be construed as limited to the embodiments set forth herein. Rather, these embodiments are provided so that this disclosure will be thorough and complete, and will fully convey the scope of the embodiments to those skilled in the art. Like reference numerals refer to like elements throughout.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

The terminology used herein is for the purpose of describing particular embodiments only and is not intended to be limiting thereof. As used herein, the singular forms “a”, “an” and “the” are intended to include the plural forms as well, unless the context clearly indicates otherwise. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another elements as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower”, can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments should not be construed as limited to the particular shapes of regions illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the disclosure.

All methods described herein can be performed in a suitable order unless otherwise indicated herein or otherwise clearly contradicted by context. The use of any and all examples, or exemplary language (e.g., “such as”), is intended merely to better illustrate the disclosure and does not pose a limitation on the scope thereof unless otherwise claimed. No language in the specification should be construed as indicating any non-claimed element as essential to the practice of the embodiments as used herein.

Hereinafter, the embodiments will be described in detail with reference to the accompanying drawings.

An embodiment of a method of authenticating a product according to the present disclosure includes: hybridizing a target polynucleotide that is contained in the product and includes a first target region and a second target region adjacent to the first target region with a probe polynucleotide that includes a first segment having a nucleotide sequence completely complementary to the first target region and a second segment having a nucleotide sequence completely complementary to the second target region; ligating one end of the first segment of the probe polynucleotide to one end of the second segment of the probe polynucleotide; amplifying the probe polynucleotide; and detecting the amplified polynucleotide.

The target polynucleotide that is hybridized with the probe polynucleotide includes the first target region and the second target region adjacent to the first target region.

The term “polynucleotide” as used herein refers to a polymer of a single-stranded or double-stranded deoxyribonucleotide or ribonucleotide. In addition, the polynucleotide includes natural polynucleotide analogues unless otherwise stated.

According to the method, the target polynucleotide is a subject to be encoded and contained in a product to be authenticated or externally attached to the product. For example, if the product is a drug in a liquid state, the target polynucleotide may be contained in the drug in the liquid state. If the product is a capsule pharmaceutical, the target polynucleotide may be attached to the surface of the capsule. Alternative configurations are also possible.

In the target polynucleotide, the first and second target regions are used as codes, e.g., for product identification. For example, in one embodiment the first and second target regions may include 2 to 60 nucleotides, respectively. Alternative embodiments may include configurations wherein the first and second target regions may also include 3 to 40 nucleotides, respectively. Additional embodiments may include configurations wherein the first and second target regions may also include 4 to 20 nucleotides, respectively. Embodiments may also include configurations wherein the first and second target regions may include more than 60 nucleotides.

The authentication of the product may be performed by decoding the first and second target regions. In addition, the number of codes may vary according to those of ordinary skill in the art within the range of the number of nucleotides described above. For example, in an embodiment wherein the nucleotide is deoxyribonucleotide and the first and second target regions respectively include 5 nucleotides, 4¹⁰ codes may be used. Thus, a larger or smaller number of codes for the products to be authenticated may be obtained by changing the number of the nucleotides of the first and second target regions.

An embodiment of the target polynucleotide may include 20 to 200 nucleotides. Embodiments also include configurations wherein the target polynucleotide may include 30 to 150 nucleotides. In an alternative embodiment, the target polynucleotide may also include 40 to 100 nucleotides.

Since the target polynucleotide functions as a code for authenticating the product, the target polynucleotide is prepared so as not to be readily copied by a third party. That is, the target polynucleotide is prepared such that the sequence of the target regions cannot be analyzed by inhibiting the generation of a hydroxyl group at 3′-end of the target polynucleotide. Thus, the 3′-hydroxyl group of the target polynucleotide may be substituted with a functional group selected from the group consisting of an amino group, a nitro group, an aldehyde group, an alkyl group, an allyl group, an aryl group, and a phenyl group. For the same reason, the 5′-phosphate group of the target polynucleotide may also be substituted with a functional group selected from the group consisting of an amino group, a nitro group, an aldehyde group, an alkyl group, an allyl group, an aryl group, and a phenyl group. Furthermore, all of the phosphodiester bonds in the target polynucleotide may be deformed into bonds that are not cleaved by endonuclease, thus prohibiting the use of endonuclease in copying the target polynucleotide. The bonds that are not cleaved by endonuclease may be phosphorothioate bonds, boranophosphate bonds, methylphosphonate bonds, phosphorodithioate bonds, phosphoramidothioate bonds, phosphoramidite bonds, phosphordiamidate bonds, alkyl phosphotriester bonds, formacetal bonds or any combination thereof, but are not limited thereto.

The probe polynucleotide that is hybridized with the target polynucleotide includes the first and second segments.

According to the method, the probe polynucleotide decodes the target polynucleotide and may be contained in an apparatus for authenticating a product which will be described later. The first and second segments of the probe polynucleotide are nucleotides having sequences respectively corresponding to the first and second target regions. The first segment has a nucleotide sequence completely complementary to the first target region and a second segment has a nucleotide sequence completely complementary to the second target region. That is, in order to decode the first and second target regions, all nucleotides of the first segment form completely complementary bonds with all nucleotides of the first target region without a mismatch, and all nucleotides of the second segment also form completely complementary bonds with all nucleotides of the second target region without a mismatch.

The first and second segments may respectively include a 5′-end or 3′-end nucleotide and 2 to 30 consecutive nucleotides from the 5′-end or 3′-end nucleotide. In one embodiment, the first and second segments may also include 3 to 20 consecutive nucleotides, respectively. In another embodiment, the first and second segments may also include 4 to 10 consecutive nucleotides, respectively. That is, the first and second segments may be positioned at both ends of the probe polynucleotide, respectively.

In one embodiment, the probe polynucleotide may include 10 to 300 nucleotides. Embodiments also include configurations wherein the probe polynucleotide may include 20 to 200 nucleotides. In another embodiment the target polynucleotide may include 30 to 100 nucleotides. In addition, the target polynucleotide and the probe polynucleotide may be single-stranded polynucleotides, respectively. The probe polynucleotide is not limited to including 10 to 300 nucleotides and alternative embodiments may contain a greater or lesser number.

In this process, the target polynucleotide may be contacted with the probe polynucleotide for the hybridization. If a capsule pharmaceutical is to be authenticated, the surface of the capsule pharmaceutical to which the target polynucleotide is attached is contacted with an apparatus for authenticating a product including the probe polynucleotide to perform the hybridization. The apparatus will be described in more detail below.

The method includes ligating one end of the first segment of the probe polynucleotide to one end of the second segment of the probe polynucleotide.

The hybridized polynucleotide may include a nick, as defined in more detail below, between the end of the first segment and the end of the second segment. That is, in the hybridized polynucleotide, the probe polynucleotide has a nick.

The term “nick” used herein refers to a single phosphodiester interruption in one of the two strands of a double-stranded polynucleotide. A nick may exist in the hybridized polynucleotide if the first and second target regions of the target polynucleotide are completely hybridized with the first and second segments of the probe polynucleotide so that the 5′-end or 3′-end nucleotide of the first segment is adjacent to the 3′-end or 5′-end nucleotide of the second segment.

The ligating of the first and second segments indicates ligating the nick. That is, the ligating is conducted by recognizing the nick in the probe polynucleotide of the hybridized double-stranded polynucleotide that is obtained by hybridizing the target polynucleotide with the probe polynucleotide and forming a phosphodiester bond in the nick. The ligating process may be conducted by a ligase such as T4 DNA ligase or other similar ligases.

The probe polynucleotide may be shaped to have a single-stranded circular form via the ligating process.

The method also includes amplifying the probe polynucleotide.

The term “amplification” used herein refers to a procedure for producing multiple copies of polynucleotides complementary to a single-stranded template polynucleotide via polymerization of nucleotides. If the amplification is performed by the polymerization of DNA, a primer, as is known in the art, may be added to the amplification. The primer may be an oligonucleotide including 5 to 30 nucleotides and complementary to the probe polynucleotide or an oligonucleotide including 6 random nucleotides (also referred to as a “random hexamer”).

In one embodiment, the amplification may be conducted by polymerase chain reaction (“PCR”), ligase chain reaction, transcription-mediated amplification, selective amplification of target polynucleotide sequence, consensus sequence primed polymerase chain reaction (“CP-PCR”), arbitrarily primed polymerase chain reaction (“AP-PCR”), nucleic acid based sequence amplification (“NABSA”), rolling circle amplification, multiple displacement amplification, or circle-to-circle amplification. In addition, the amplification may be conducted under conditions that are well known in the art, generally, at room temperature, although the present disclosure is not limited thereto.

The method includes detecting the amplified polynucleotide.

In order to detect the amplified polynucleotide, the amplified polynucleotide may include a detectable label.

The term “detectable label” used herein refers to an atom or molecule used to specifically detect a molecule including the label among the same type of molecules without the label. For example, embodiments of the detectable label may include colored bead, antigen determinant, enzyme, hybridizable nucleic acid, chromophore, fluorescent material, phosphorescent material, electrically detectable molecule, a molecule providing modified fluorescence-polarization or modified light-diffusion, a quantum dot, and other similar elements. In addition, embodiments of the detectable label may be radioactive isotopes such as P³², S³⁵, or other such isotopes, a chemiluminescent compound, a labeled binding protein, a heavy metal atom, a spectroscopic marker such as a dye, a magnetic label or other similar elements.

Embodiments of the dye may be quinoline dye, triarylmethane dye, phthalene, azo dye, or cyanine dye, but the dye is not limited thereto. Embodiments of the fluorescent material may be fluorescein, phycoerythrin, rhodamine, lissamine, or Cy3 or Cy5 (Pharmacia), but the fluorescent material is not limited thereto.

The detectable label may be contained in the amplified polynucleotide, i.e., amplified probe polynucleotide, so that the amplified polynucleotide may be selectively detected. For example, if the fluorescent material is used as the detectable label, the fluorescence may be detected using a scanner that detects the fluorescence.

The method may further include determining the product as an authenticated product if the amplified polynucleotide is detected via the detecting process.

Another embodiment of an apparatus for authenticating a product according to the present disclosure includes: a sample inlet including the probe polynucleotide and a ligase; an amplification unit including a polynucleotide polymerase, a primer, and dNTP; and a detection unit that detects the amplified polynucleotide.

In the sample inlet, the target polynucleotide is contacted with the probe polynucleotide as described above with reference to the method of authenticating a product. Since the nick between the first and second segments of the probe polynucleotide is ligated in the sample inlet, the ligation is conducted in a liquid state. Thus, the sample inlet may include a buffer solution that is known in the art so that the ligase may function.

The amplification unit in which the probe polynucleotide is amplified includes a polynucleotide polymerase, a primer, and dNTP. Thus, the amplification unit may include a buffer solution that is known in the art so that the polynucleotide polymerase may be mobile. The buffer solution contained in the amplification unit may be compatibly used with the buffer solution contained in the sample inlet. In this regard, the buffer solution contained in the sample inlet may be transferred to the amplification unit and used. Meanwhile, the dNTP may further include a detectable label. The detectable label, as described above, may be selected from the group consisting of a colored bead, an antigen determinant, an enzyme, a chromophore, a fluorescent material, a phosphorescent material, an electrically detectable molecule, a molecule providing modified fluorescence-polarization or modified light-diffusion, a quantum dot and other similar elements.

The detection unit is a unit that detects the amplified probe polynucleotide. The detection unit may further include an interchelator that is specifically incorporated into the polynucleotide. For example, embodiments of the interchelator may include Alexa Fluor 350®, Alexa Fluor 430®, Alexa Fluor 488®, Alexa Fluor 532®, Alexa Fluor 546®, Alexa Fluor 568®, Alexa Fluor 594®, Alexa Fluor 633®, Alexa Fluor 647®, Alexa Fluor 660®, Alexa Fluor 680®, Cy2®, Cy3.18, Cy3.5®, Cy3®, Cy5.18, Cy5.5®, Cy5®, Cy7®, Oregon Green, Oregon Green 488-X, Oregon Green®, Oregon Green®488, Oregon Green®500, Oregon Green®514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3, thiazole orange, ethidium bromide and other similar elements, but the interchelator is not limited thereto. For example, in an embodiment wherein the detection unit includes ethidium bromide, ethidium bromide is incorporated into the amplified probe polynucleotide that is transferred to the detection unit, and thus the amplified polynucleotide may efficiently be detected using ultra-violet (“UV”) rays.

Meanwhile, the sample inlet, the amplification unit, and the detection unit may be connected to each other to allow fluid flow therebetween. In addition, the apparatus may further include a filter that filters the ligase at a connection region between the sample inlet and the amplification unit so as to prevent the migration of the ligase that is contained in the sample inlet to the amplification unit. In addition, embodiments include configurations wherein the apparatus may be manufactured by fixing the sample inlet, the amplification unit, and the detection unit to a solid support such as a micro plate for convenience of use.

The present invention will be described in further detail with reference to the following examples. These examples are for illustrative purposes only and are not intended to limit the scope of the invention.

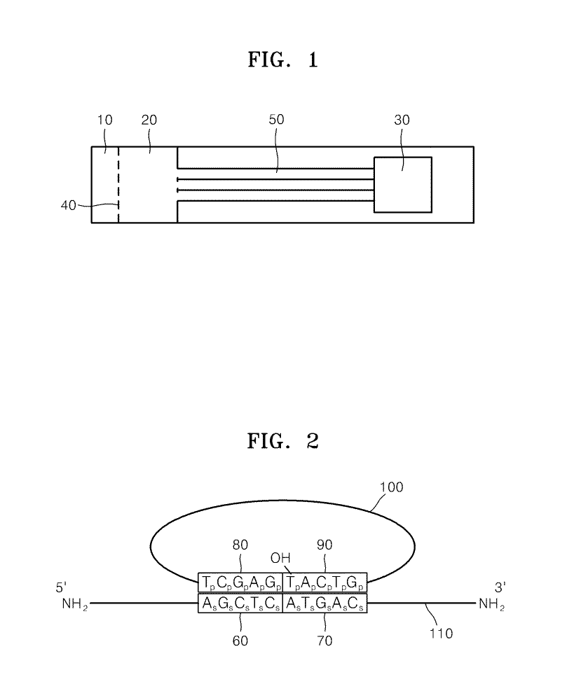

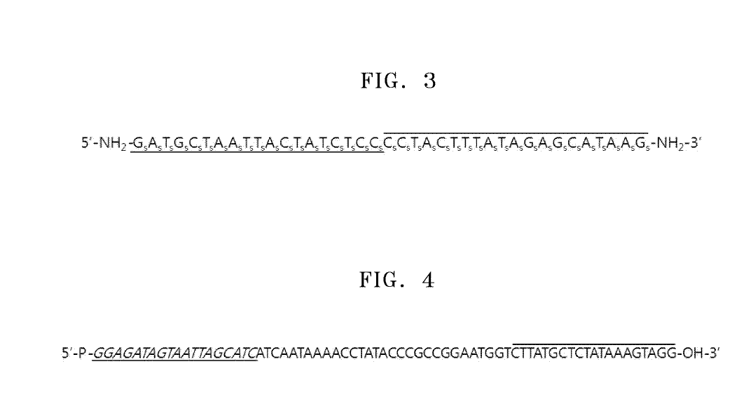

FIG. 1 schematically illustrates an embodiment of an apparatus for authenticating a product according to the present disclosure. FIG. 2 schematically illustrates a hybridization reaction according to an embodiment of a method of authenticating a product according to the present disclosure. Referring to FIGS. 1 and 2, a method of authenticating a product using a polynucleotide will be described.

First, a target polynucleotide 110 contained in a product to be authenticated is contacted with a sample inlet 10 including a probe polynucleotide 100 and a ligase. In one embodiment, the target polynucleotide 110 may be contained in a buffer solution that may stabilize the target polynucleotide 110 is known in the art. Alternative embodiments include configurations wherein the target polynucleotide 110 may be in a dry state. In such an alternative embodiment, the sample inlet 10 may not include the buffer solution.

If the product to be authenticated is a genuine product, the first target region 60 and the second target region 70 of the target polynucleotide 110 are hybridized, i.e., completely hybridized, with the first segment 80 and the second segment 90 of the probe polynucleotide 100, and a nick is formed between the first segment 80 and the second segment 90 of the hybridized double-stranded polynucleotide in the sample inlet 10 as described above. The ligase contained in the sample inlet 10 ligates the nick, and the probe polynucleotide 100, which in the present embodiment is a single-stranded circular polynucleotide obtained by ligating the nick is transferred to the amplification unit 20 via pores of a filter 40. In this regard, the ligase is filtered by the filter 40 so as not to be transferred to the amplification unit 20. The probe polynucleotide 100 that is transferred to the amplification unit 40 with the buffer solution is amplified via rolling circle amplification using the polynucleotide polymerase, a primer, and dNTP that are contained in the amplification unit 20.

The amplified polynucleotides are transferred to the detection unit 30 that contains a detectable label with the buffer solution via a migration channel 50 that is connected to allow fluid flow between the amplification unit 20 and the detection unit 30. Then, an interchelator that is specifically incorporated into polynucleotide and used as a detectable label, for example, ethidium bromide, is incorporated into the amplified polynucleotides that are transferred to the detection unit 30. If UV rays are irradiated to the detection unit 30, the existence of the amplified polynucleotides and the intensity of the signal may be identified, so that the authenticity of a product may be identified.

The following examples show the results of authenticating a product using the above embodiment of an apparatus for authenticating a product according to the method.

### EXAMPLE 1

### Preparation of Target Polynucleotide and Probe Polynucleotide

A target polynucleotide and probe polynucleotide were purchased from Bioneer Corp., Ltd. A 39-mer target polynucleotide was prepared, and a 69-mer probe polynucleotide was prepared. All of the phosphodiester bonds in the target polynucleotide were deformed into phosphorothioate bonds. All polynucleotides were synthesized using a scale with 0.2 μM, and quality test thereof was performed using MALDI-TOF after PAGE purification.

FIG. 3 illustrates a sequence of a target polynucleotide used in Example 1 according to an embodiment of the present invention. The 5′-end and 3′-end in the target polynucleotide are substituted with amine groups. In addition, the sequence of the target polynucleotide includes a first target sequence, which is underlined in FIG. 3, including 19 consecutive nucleotides from the 5′-end nucleotide and a second target sequence, which is overlined in FIG. 3, including 20 consecutive nucleotides from the next nucleotide to the first target sequence as shown in FIG. 3.

FIG. 4 illustrates a sequence of a probe polynucleotide used in Example 1. As shown in FIG. 4, the sequence of the probe polynucleotide includes a first segment sequence, which is underlined in FIG. 4, including 19 consecutive nucleotides from the 5′-end nucleotide, a second segment sequence, which is overlined in FIG. 4, including 20 consecutive nucleotides from the 3′-nucleotide, and a sequence including 30 consecutive nucleotides that form a loop between the first and second segments.

The sequences of the target polynucleotide and the probe polynucleotide are respectively shown in sequence lists (SEQ ID NOS: 1 and 2).

### EXAMPLE 2

### Authentication of Product Using the Target Polynucleotide and the Probe Polynucleotide

The target polynucleotide (1 fM) and the probe polynucleotide (1 pM) prepared according to Example 1 were added to 20 μl of a buffer solution [50 mM Tris-HCl, 10 mM MgCl₂, 1 mM ATP, and 10 mM dithiothreitol; pH 7.5], and hybridized at 25° C. for 1 to 2 minutes. Then, 1 μl of a T4 DNA ligase (Promega) was added to the mixture, and the mixture was maintained at 37° C. for 10 minutes. Then, amplification was performed using 1 μl of the resultant mixture. The amplification was conducted using RepliG-ultra Fast mini KIT (Qiagen, cat No. 150035), and all experiments were conducted according to protocols of the kit. Here, SYBR® Green II was used as a detectable label according to the protocols. The amplification was performed using TMC-1000 (Samsung Advanced Institute of Technology), as a Real-time PCR device, under isothermal conditions of 30° C. for 115 seconds, and then fluorescent detection was repeated for 5 seconds. This test was repeated four times.

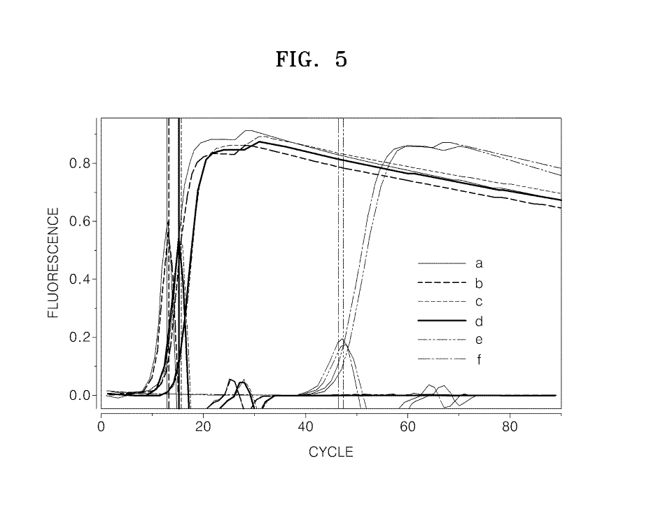

FIG. 5 is a graph illustrating the results of the amplification. In the graph of FIG. 5, first four curves (a, b, c and d) indicate the results obtained by repeating the test four times, and second two curves (e and f) indicate results of control groups to which the target polynucleotide was not added. According to the results, the amplification was performed within 20 to 40 minutes when using 1 fM of target polynucleotide, while the amplification was performed after 80 minutes in the control groups. Since the amplification is sensitively performed using the RepliG-ultra Fast mini KIT (Example 2), non-specific amplification may occur even though there is no subject. However, this amplification occurs after a long period of time, and thus the existence of the target may be identified prior to the non-specific amplification. Thus, it was identified that authenticating a product may be conducted using the target polynucleotide and the probe polynucleotide using the amplification.

As described above, according to the one or more of the above embodiments of the present invention, genuine products and counterfeit products may be efficiently distinguished from each other using the apparatus and method of authenticating a product as described above.

It should be understood that the embodiments described therein should be considered in a descriptive sense only and not for purposes of limitation. Descriptions of features or aspects within each embodiment should typically be considered as available for other similar features or aspects in other embodiments.

What is claimed is:

1. A method of authenticating a product, the method comprising:
hybridizing a target polynucleotide which is associated with the product and which comprises a first target region and a second target region adjacent to the first target region with a probe polynucleotide that comprises a first segment having a nucleotide sequence completely complementary to the first target region and a second segment having a nucleotide sequence completely complementary to the second target region;
ligating one end of the first segment of the probe polynucleotide to one end of the second segment of the probe polynucleotide;
amplifying the probe polynucleotide; and
detecting the amplified polynucleotide.

2. The method of claim 1, wherein the first target region and the second target region respectively comprise about 2 nucleotides to about 60 nucleotides.

3. The method of claim 1, wherein the target polynucleotide comprises about 2 to about 200 nucleotides.

4. The method of claim 1, wherein a 3′-hydroxyl group of the target polynucleotide is substituted with a functional group selected from the group consisting of an amino group, a nitro group, an aldehyde group, an alkyl group, an allyl group, an aryl group and a phenyl group.

5. The method of claim 1, wherein all of the phosphodiester bonds in the target polynucleotide are deformed into bonds that are not cleaved by endonuclease.

6. The method of claim 1, wherein the first segment and the second segment respectively comprise one of a 5′-end and a 3′-end nucleotide and about 2 to about 30 consecutive nucleotides from the 5′-end or 3′-end nucleotide.

7. The method of claim 1, wherein the probe polynucleotide comprises about 10 to about 300 nucleotides.

8. The method of claim 1, wherein the target polynucleotide and the probe polynucleotide are single-stranded.

9. The method of claim 1, wherein the hybridized polynucleotide comprises a nick between an end of the first segment and an end of the second segment.

10. The method of claim 1, wherein the ligating is performed by a ligase.

11. The method of claim 1, wherein the amplifying is performed by one of rolling circle amplification and multiple displacement amplification.

12. The method of claim 1, wherein the amplifying is performed at room temperature.

13. The method of claim 1, wherein the amplified polynucleotide comprises a detectable label.

14. The method of claim 1, further comprising determining the product as an authenticated product if the amplified polynucleotide is detected during the detecting.

15. An apparatus for authenticating a product, the apparatus comprising:
a sample inlet comprising a probe polynucleotide that comprises a first segment having a nucleotide sequence completely complementary to a first target region of a target polynucleotide and a second segment having a nucleotide sequence completely complementary to a second target region of a target polynucleotide and a ligase, wherein the first target region and the second target region of the target polynucleotide are adjacent to one another;
an amplification unit comprising a polynucleotide polymerase, a primer, and dNTP; and
a detection unit which detects the amplified polynucleotides.

16. The apparatus of claim 15, wherein the detection unit further comprises an interchelator which is specifically incorporated into the polynucleotides.

17. The apparatus of claim 16, wherein the interchelator is one of SYBR Green and ethidium bromide.

18. The apparatus of claim 15, wherein the dNTP further comprises a detectable label.

19. The apparatus of claim 18, wherein the detectable label comprises one selected from the group consisting of a colored bead, an antigen determinant, an enzyme, a chromophore, a fluorescent material, a phosphorescent material, an electrically detectable molecule, a molecule providing modified fluorescence-polarization or modified light-diffusion, and a quantum dot.

20. The apparatus of claim 15, wherein the sample inlet, the amplification unit, and the detection unit are fluidly connected to each other to allow fluid flow therebetween.


SEQUENCE LISTING (2 sequences)

SEQ ID NO: 1 - DNA, 39 bp, Artificial Sequence
  target polynucleotide for authenticating product
gatgctaattactatctcccctactttatagagcataag

SEQ ID NO: 2 - DNA, 69 bp, Artificial Sequence
  probe polynucleotide for authenticating product
ggagatagtaattagcatcatcaataaaacctatacccgccggaatggtcttatgctctataaagtagg